(19) United States
(12) Patent Application Publication
(10) Pub. No.: US 2008/0248110 A1
(43) Pub. Date: Oct. 9, 2008

(54) Pharmaceutical Compositions Useful in the Treatment of Pain

(76) Inventors: Anders Pettersson, Uppsala (SE); Thomas Lundqvist, Uppsala (SE)

Correspondence Address: MORGAN LEWIS & BOCKIUS LLP, 1111 PENNSYLVANIA AVENUE NW, WASHINGTON, DC, 20004, US

(21) Appl. No.: 11/885,159

(22) PCT Filed: Mar. 28, 2006
(86) PCT No.: PCT/GB2006/001133

§ 371 Date: Jun. 6, 2008

Related U.S. Application Data
(60) Provisional application No. 60/665,377, filed on Mar. 28, 2005.

(51) Int. Cl. A61K 9/14; A61K 31/445; A61K 31/4178; A61K 31/485; A61K 9/20; A61P 25/04

(52) U.S. Cl. 424/465; 424/489; 514/329; 514/397; 514/289; 514/282

(57) ABSTRACT

There is provided pharmaceutical compositions for the treatment of pain comprising a pharmacologically-effective amount of an opioid analgesic, or a pharmaceutically-acceptable salt thereof; a pharmacologically-effective amount of an antiemetic compound, or a pharmaceutically-acceptable salt thereof; a bioadhesion and/or a mucoadhesion promoting agent; and carrier particles, wherein the active ingredients are presented in particulate form upon the surfaces of the carrier particles, which carrier particles are larger in size than the particles of the active ingredients; and the bioadhesion and/or mucoadhesion promoting agent is, at least in part, presented on the surfaces of the carrier particles.

This invention relates to new, fast acting pharmaceutical compositions that are useful in the treatment of pain, which compositions may be administered transmucosally and in particular sublingually.

Opioids are widely used in medicine as analgesics. Indeed, it is presently accepted that, in the palliation of more severe pain, no more effective therapeutic agents exist.

The term “opioid” is typically used to describe a drug that activates opioid receptors, which are found in the brain, the spinal cord and the gut. Three classes of opioids exist:
(a) naturally-occurring opium alkaloids. These include morphine and codeine;
(b) compounds that are similar in their chemical structure to the naturally-occurring opium alkaloids. These so-called semi-synthetics are produced by chemical modification of the latter and include the likes of diamorphine (heroin), oxycodone and hydrocodone; and
(c) truly synthetic compounds such as fentanyl and methadone. Such compounds may be completely different in terms of their chemical structures to the naturally-occurring compounds.

Of the three major classes of opioid receptors (μ, κ and δ), opioids' analgesic and sedative properties derives from agonism at the μ receptor.

Opioid analgesics are used to treat the severe, chronic pain of terminal cancer, often in combination with non-steroidal anti-inflammatory drugs (NSAIDs), as well as acute pain (e.g. during recovery from surgery). Further, their use is increasing in the management of chronic, non-malignant pain.

Opioid-requiring cancer patients are usually given slow-release opiates (slow-release morphine or ketobemidone, or transdermal fentanyl). A characteristic feature of such treatments is periods of inadequate analgesia (so-called “breakthrough” pain). Such periods are thought to be due to increased physical activity of the patient. However, treatment of breakthrough pain by administration of increased time-contingent doses of long-acting analgesic formulations is known to cause adverse side effects, including excess sedation, nausea, and constipation.

Presently-available oral, rectal and sublingual opioid analgesic formulations have relatively lengthy onset times and/or erratic absorption characteristics, which makes then not entirely suitable for the control of acute and/or breakthrough pain.

In order to obtain rapid onset of analgesia in the treatment of other types of acute pain, including operative pain, post-operative pain, traumatic pain, post-traumatic pain, and pain caused by severe diseases, such as myocardial infarction, nephrolithiasis, etc., opioid analgesics are often administered parenterally (e.g. by intravenous or intramuscular injection). However, injections are an unpopular mode of administration, often being regarded as inconvenient and painful.

In view of the above, there is a real and growing clinical need for fast-acting orally-delivered drug compositions comprising opioid analgesics. In particular, a need exists for further or better fast-acting formulations comprising opioid analgesics, which may be administered by a convenient route, for example transmucosally, particularly, as is usually the case, when such active ingredients are incapable of being delivered perorally due to poor and/or variable bioavailability.

International patent applications WO 00/16750 and WO 2004/067004 disclose drug delivery systems for the treatment of acute disorders by e.g. sublingual administration, in which the active ingredient is in microparticulate form and is adhered to the surfaces of larger carrier particles in the presence of a bioadhesive and/or mucoadhesive promoting agent. Specific combinations of opioid analgesics and antiemetic agents are not mentioned or suggested anywhere in these documents.

According to a first aspect of the invention there are provided particulate pharmaceutical compositions for the treatment of pain comprising:
(a) a pharmacologically-effective amount of an opioid analgesic, or a pharmaceutically-acceptable salt thereof;
(b) a pharmacologically-effective amount of an antiemetic compound, or a pharmaceutically-acceptable salt thereof;
(c) a bioadhesion and/or a mucoadhesion promoting agent; and
(d) carrier particles,
   wherein
(1) active ingredients (a) and (b) are presented in particulate form upon the surfaces of the carrier particles, which carrier particles are larger in size than the particles of the active ingredients; and
(2) the bioadhesion and/or mucoadhesion promoting agent is, at least in part, presented on the surfaces of the carrier particles,
   which compositions are referred to hereinafter as “the compositions of the invention”.

The compositions of the invention are interactive mixtures. The term “interactive” mixture will be understood by those skilled in the art to denote a mixture in which particles do not appear as single units, as in random mixtures, but rather where smaller particles (of, for example, active ingredient(s) or bioadhesion and/or mucoadhesion promoting agent) are attached to (i.e. adhered to or associated with) the surfaces of larger carrier particles. Such mixtures are characterised by interactive forces (for example van der Waals forces, electrostatic or Coulombic forces, and/or hydrogen bonding) between carrier and surface-associated particles (see, for example, Staniforth, Powder Technol., 45, 73 (1985)). In the final mixture, the interactive forces need to be strong enough to keep the adherent particles at the carrier surface, in order to create a homogeneous mixture.

The term “pharmacologically effective amount” refers to an amount of an active ingredient, which is capable of conferring a desired therapeutic effect on a treated patient, whether administered alone or in combination with another active ingredient. Such an effect may be objective (i.e. measurable by some test or marker) or subjective (i.e. the subject gives an indication of, or feels, an effect).

The term “opioid analgesic” will be understood by the skilled person to include any substance, whether naturally-occurring or synthetic, with opioid or morphine-like properties and/or which binds to opioid receptors, particularly the g-opioid receptor, having at least partial agonist activity, thereby capable of producing an analgesic effect.

Opioid analgesics that may be mentioned include opium derivatives and the opiates, including the naturally-occurring phenanthrenes in opium (such as morphine, codeine, thebaine and Diels-Alder adducts thereof) and semisynthetic derivatives of the opium compounds (such as diamorphine, hydromorphone, oxymorphone, hydrocodone, oxycodone, etorphine, nicomorphine, hydrocodeine, dihydrocodeine, metopon, normorphine and N-(2-phenylethyl)normorphine). Other opioid analgesics that may be mentioned include fully synthetic compounds with opioid or morphine-like properties, including morphinan derivatives (such as racemorphan, levorphanol, dextromethorphan, levallorphan, cyclorphan, butorphanol and nalbufine); benzomorphan derivatives (such as cyclazocine, pentazocine and phenazocine); phenylpiperidines (such as pethidine (meperidine), fentanyl, alfentanil, sufentanil, remifentanil, ketobemidone, carfentanyl, anileridine, piminodine, ethoheptazine, alphaprodine, betaprodine, 1-methyl-4-phenyl-1,2,3,6-tetrahydropyridine (MPTP), diphenoxylate and loperamide), phenylheptamines or “open chain” compounds (such as methadone, isomethadone, propoxyphene and levomethadyl acetate hydrochloride (LAAM)); diphenylpropylamine derivatives (such as dextromoramide, piritramide, bezitramide and dextropropoxyphene); mixed agonists/antagonists (such as buprenorphine, nalorphine and oxilorphan) and other opioids (such as tilidine, tramadol and dezocine). More preferred opioid analgesics include buprenorphine and fentanyl.

Preferred antiemetics include phenothiazines, such as prochlorperazine, metopimazine, thiethylperazine, alimenazine, promethazine and chlorpromazine; 5-HT₃ antagonists, such as ondansetron, granisetron, tropisetron, azasetron, dolasetron and ramosetron; dopamine receptor antagonists, such as metoclopramide, clebopride, alizapride, bromopride, itopride and domperidone; antihistamines, such as dimenhydrinate, doxylamine, diphenhydramine, buclizine and cyclizine, and piperazine derivatives, such as ceterazine and meclizine; butyrophenones, such as haloperidol and droperidol; cannabinoids, such as dronabinol, levonantradol and nabilone; antichlolinergics, such as difenidol; and other drugs, such as cerium oxalate and ginger. More preferred antiemetics include phenothiazines, antihistamines and 5-HT₃ receptor antagonists, especially ondansetron and granisetron.

Any of the active ingredients mentioned in the above groupings may also be used in combination as required. Moreover, the above active ingredients may be used in free form or, if capable of forming salts, in the form of a salt with a suitable acid or base. If the drugs have a carboxyl group, their esters may be employed. Active ingredients can be used as racemic mixtures or as single enantiomers.

The active ingredients in the compositions of the invention are preferably in the form of microparticles, preferably with a weight based mean diameter of between about 0.5 μm and about 15 μm, such as about 1 μm and about 10 μm. The term “weight based mean diameter” will be understood by the skilled person to include that the average particle size is characterised and defined from a particle size distribution by weight, i.e. a distribution where the existing fraction (relative amount) in each size class is defined as the weight fraction, as obtained e.g. by sieving.

Microparticles of active ingredients may be prepared by standard micronisation techniques, such as grinding, dry milling, wet milling, precipitation, etc.

The amounts of active ingredients that may be employed in compositions of the invention may be determined by the physician, or the skilled person, in relation to what will be most suitable for an individual patient. This is likely to vary with the route of administration, the type and severity of the condition that is to be treated, as well as the age, weight, sex, renal function, hepatic function and response of the particular patient to be treated.

The total amount of active ingredients (a) and (b) that may be employed in a composition of the invention may be in the range 0.1 (e.g. 1, such as 2) to 20% by weight based upon the total weight of the composition. More preferably, compositions of the invention may contain between 4 and 17% by weight of active ingredients, and especially from about 5 to about 15%. The amount(s) of active ingredients may also be expressed as the amount(s) of active ingredients in a unit dosage form (e.g. a tablet). In such a case, the amount of active ingredients that may be present may be sufficient to provide a dose per unit dosage form that is in the range of between about 5 μg and about 20 mg of active ingredients in total.

The above-mentioned dosages are exemplary of the average case; there can, of course, be individual instances where higher or lower dosage ranges are merited, and such are within the scope of this invention.

It is possible for certain active ingredients that the relative sizes and amounts of the particles of active ingredients and the carrier particles that are employed are sufficient to ensure that the carrier particles may be at least about 90% covered by the active ingredients, for, example at least about 100% and up to about 200% (e.g. between about 130% and about 180%) covered. The skilled person will appreciate in this context that “100% coverage” of the carrier particles by the active ingredients means that the relative particle sizes and amounts of the relevant particles that are employed are sufficient to ensure that the entire surface area of each carrier particle could be covered by particles of the active ingredients notwithstanding that other ingredients (e.g. mucoadhesion promoting agent) may also be present in a composition. Obviously, if other such ingredients are employed, then the actual degree of coverage of carrier particles by active ingredients may be less than the amounts specified above. 200% coverage means that there is sufficient particles of active ingredients to cover the surfaces of the carrier particles twice over, notwithstanding the presence of other ingredients.

It is surprising that compositions with greater than 90% theoretical coverage are effective. Based on current knowledge, the skilled person would understand that, in order to ensure rapid dissolution, it would be important to ensure that the relative sizes/amounts of active ingredients/carrier particles are sufficient to ensure that 70% or less of the surfaces of the latter could be covered by the former.

Compositions of the invention comprise one or more bioadhesion and/or mucoadhesion promoting agent and may thus facilitate the partial or complete adhesion of active ingredients to a biological surface, such as a mucosal membrane.

The terms “mucoadhesive” and “mucoadhesion” refer to adhesion or adherence of a substance to a mucous membrane within the body, wherein mucous is present on the surface of that membrane (e.g. the membrane is substantially (e.g. >95%) covered by mucous). The terms “bioadhesive” and “bioadhesion” refer to adhesion or adherence of a substance to a biological surface in a more general sense. Biological surfaces as such may include mucous membranes wherein mucous is not present on that surface, and/or surfaces that are not substantially (e.g. <95%) covered by mucous. The skilled person will appreciate that, for example, the expressions “mucoadhesion” and “bioadhesion” may often be used interchangeably. In the context of the present invention, the relevant terms are intended to convey a material that is capable of adhering to a biological surface when placed in contact with that surface (in the presence of mucous or otherwise) in order to enable compositions of the invention to adhere to that surface. Such materials are hereinafter referred to together as “bio/mucoadhesives” or “bio/mucoadhesion promoting agents”, and such properties together as “bio/mucoadhesion” or “bio/mucoadhesive”.

A variety of polymers known in the art can be used as bio/mucoadhesion promoting agents, for example polymeric substances, preferably with an average (weight average) molecular weight above 5,000. It is preferred that such materials are capable of rapid swelling when placed in contact with water and/or, more preferably, mucous, and/or are substantially insoluble in water at room temperature and atmospheric pressure.

Bio/mucoadhesive properties may be routinely determined in a general sense in vitro, for example as described by G. Sala et al in Proceed. Int. Symp. Contr. Release. Bioact. Mat., 16, 420, 1989. Examples of suitable bio/mucoadhesion promoting agents include cellulose derivatives such as hydroxypropylmethyl cellulose (HPMC), hydroxyethyl cellulose (HEC), hydroxypropyl cellulose (HPC), methyl cellulose, ethyl hydroxyethyl cellulose, carboxymethyl cellulose, modified cellulose gum and sodium carboxymethyl cellulose (NaCMC); starch derivatives such as moderately cross-linked starch, modified starch and sodium starch glycolate; acrylic polymers such as carbomer and its derivatives (Polycarbophyl, Carbopol®, etc.); polyvinylpyrrolidone; polyethylene oxide (PEO); chitosan (poly-(D-glucosamine)); natural polymers such as gelatin, sodium alginate, pectin; scleroglucan; xanthan gum; guar gum; poly co-(methylvinyl ether/maleic anhydride); and crosscarmellose (e.g. crosscarmellose sodium). Such polymers may be crosslinked. Combinations of two or more bio/mucoadhesive polymers can also be used.

Suitable commercial sources for representative bio/mucoadhesive polymers include: Carbopol® acrylic copolymer (BF Goodrich Chemical Co, Cleveland, 08, USA); HPMC (Dow Chemical Co., Midland, Mich., USA); NEC (Natrosol; Hercules Inc., Wilmington, Del. USA); HPC (Klucel; Dow Chemical Co., Midland, Mich., USA); NaCMC (Hercules Inc. Wilmington, Del. USA); PEO (Aldrich Chemicals, USA); sodium alginate (Edward Mandell Co., Inc., Carmel, N.Y., USA); pectin (BF Goodrich Chemical Co., Cleveland, Ohio, USA); crosslinked polyvinylpyrrolidone (Kollidon CL®, BASF, Germany, Polyplasdone XL®, Polyplasdone XL-10® and Polyplasdone INF-100, ISP Corp., US); Ac-Di-Sol® (modified cellulose gum with a high swellability; FMC Corp., USA); Actigum (Mero-Rousselot-Satia, Baupte, France); Satiaxana (Sanofi BioIndustries, Paris, France); Gantrez® (ISP, Milan, Italy); chitosan (Sigma, St Louis, Miss., USA); and sodium starch glycolate (Primojel®, DMV International BV, Netherlands, Vivastar®, J. Rettenmaier & Sohne GmbH & Co., Germany, Explotab®, Roquette America, US).

Preferred bio/mucoadhesion promoting agents that may be employed in compositions of the invention include internally crosslinked sodium carboxymethylcellulose, such as croscarmellose sodium NF (e.g. Ac-Di-Sol (FMC Corp., USA)) and, particularly, crosslinked polyvinylpyrollodine (e.g. Kollidon CL®, BASF, Germany).

Depending on the type of the bio/mucoadhesion promoting agent used, the rate and intensity of bio/mucoadhesion may be varied.

Suitably, the amount of bio/mucoadhesion promoting agent that is present in a composition of the invention may be in the range of about 0.1 to about 25% by weight based upon the total weight of the composition. A preferred range is from about 0.5 to about 15% by weight, such as about 1 to about 10% (e.g. about 2 to about 8%) by weight.

Bio/mucoadhesion promoting agent is at least in part presented on and/or adhered to the surface of a carrier particle in a composition of the invention.

Preferably, carrier particles for use in compositions of the invention are of a size that is between about 50 and about 750 μm, and preferably between about 100 and about 600 μm.

Suitable carrier particle materials that may be used comprise pharmaceutically-acceptable substances, such as carbohydrates, e.g. sugar, mannitol and lactose; pharmaceutically-acceptable inorganic salts, such as sodium chloride, calcium phosphate, dicalcium phosphate hydrate, dicalcium phosphate dehydrate, tricalcium phosphate, calcium carbonate, and barium sulfate; polymers, such as microcrystalline cellulose, cellulose and crosslinked polyvinylpyrrolidone; or mixtures thereof.

Compositions of the invention, once prepared, are preferably directly compressed/compacted into unit dosage forms (e.g. tablets) for administration to mammalian (e.g. human) patients, for example as described hereinafter.

A disintegrating agent, or “disintegrant” may also be included in the composition of the invention, particularly those that are in the form of tablets for e.g. sublingual administration. Such an agent may be defined as any material that is capable of accelerating to a measurable degree the disintegration/dispersion of a composition of the invention, and in particular carrier particles, as defined herein. This may be achieved, for example, by the material being capable of swelling and/or expanding when placed in contact with water and/or mucous (e.g. saliva), thus causing tablet formulations/carrier particles to disintegrate when so wetted. Suitable disintegrants include cross-linked polyvinylpyrrolidone, carboxymethyl starch and natural starch and mixtures thereof.

If present, disintegrating agent is preferably employed in an amount of between 0.5 and 10% by weight based upon the total weight of the composition. A preferred range is from 1 to 8%, such as from about 2 to about 7% (e.g. about 5%) by weight.

It will be evident from the list of possible disintegrants provided above that certain materials may function in compositions of the invention in the form of tablets both as bio/mucoadhesion promoting agents and as disintegrating agents. Thus, these functions may both be provided by different substances or may be provided by the same substance.

When the “same” material is employed as a bio/mucoadhesive and as a disintegrant, the material can be said to be in two separate fractions (a bio/mucoadhesive fraction and a disintegrant fraction). In such instances, it is preferred that the particles within the disintegrant fraction are coarser (i.e. are, relatively speaking, of a larger particle size) than those in the bioadhesive fraction (vide infra).

In any event, the skilled person will appreciate that, in compositions of the invention in the form of tablets, any disintegrant (or disintegrant fraction) will be largely not presented on (i.e. attached to, adhered to and/or associated with) the surfaces of the carrier particles, but rather will be largely presented (i.e. at least about 60%, such as about 70%, e.g. about 80% and, more particularly, about 90% by weight presented) between such particles. Conversely, bio/mucoadhesive (or bio/mucoadhesive fraction) is always largely associated (i.e. is at least about 60%, such as about 70%, e.g. about 80% and, more particularly, about 90% by weight associated) with the carrier particles, that is to say presented on (i.e. attached to, adhered to and/or associated with) the surfaces of the carrier particles, or presented within such particles (vide infra), or both.

Compositions of the invention in the form of tablets for e.g. sublingual administration may also comprise a binder. A binder may be defined as a material that is capable of acting as a bond formation enhancer, facilitating the compression of the powder mass into coherent compacts. Suitable binders include cellulose gum and microcrystalline cellulose. If present, binder is preferably employed in an amount of between 0.5 and 20% by weight based upon the total weight of the tablet formulation. A preferred range is from 1 to 15%, such as from about 2.0 to about 12% (e.g. about 10%) by weight.

Compositions of the invention may comprise a pharmaceutically acceptable surfactant or wetting agent, which may enhance the hydration of active ingredients and carrier particles, resulting in faster initiation of both bio/mucoadhesion and dissolution. If present, the surfactant should be provided in finely dispersed form and mixed intimately with the active ingredients. Examples of suitable surfactants include sodium lauryl sulphate, lecithin, polysorbates, bile acid salts and mixtures thereof. If present, the surfactant may comprise between about 0.3 and about 5% by weight based upon the total weight of the composition, and preferably between about 0.5 and about 3% by weight.

Suitable further additives and/or excipients that may be employed in compositions of the invention, in particular those in the form of tablets for e.g. sublingual administration may comprise:
(a) lubricants (such as sodium stearyl fumarate or, preferably, magnesium stearate). When a lubricant is employed it should be used in very small amounts (e.g. up to about 3%, and preferably up to 2%, by weight based upon the total weight of the tablet formulation);
(b) flavourings (e.g. lemon, menthol or, preferably, peppermint powder), sweeteners (e.g. neohesperidin) and dyestuffs;
(c) antioxidants, which may be naturally occurring or otherwise (e.g. vitamin C, vitamin E, β-carotene, uric acid, uniquion, SOD, glutathione peroxidase or peroxidase catalase); and/or
(d) other ingredients, such as carrier agents, preservatives and gliding agents.

Compositions of the invention may be prepared by standard techniques, and using standard equipment, known to the skilled person.

For example, bio/mucoadhesion promoting agent may be admixed with carrier particles in several ways. In one embodiment, bio/mucoadhesion promoting agent in fine particulate form is mixed together with coarse carrier for a sufficient time in order to produce an ordered or interactive mixture. This results in discrete particles of bio/mucoadhesion promoting agent being presented on and/or adhered to the surfaces of the carrier particles. The skilled person will appreciate that, in order to obtain a dry powder formulation in the form of an interactive mixture, larger carrier particles must be able to exert enough force to break up agglomerates of smaller particles. This ability will primarily be determined by particle density, surface roughness, shape, flowability and, particularly, relative particle sizes.

The bio/mucoadhesion promoting agent suitably has a particle size with a weight based mean diameter of between about 0.1 and about 100 μm (e.g. about 1 and about 50 μm).

Particles of active ingredients may be dry mixed with carrier particles over a period of time that is sufficiently long to enable appropriate amounts of active ingredients to adhere to the surface of the carrier particles (with or without the presence of bio/mucoadhesion promoting agent). Standard mixing equipment may be used in this regard. The mixing time period is likely to vary according to the equipment used, and the skilled person will have no difficulty in determining by routine experimentation a suitable mixing time for a given combination of active ingredients, bio/mucoadhesion promoting agent and carrier particle material.

Other ingredients (e.g. disintegrants and surfactants) may be incorporated by standard mixing as described above for the inclusion of active ingredients.

The compositions of the invention may be administered transmucosally, such as buccally, rectally, nasally or preferably sublingually by way of appropriate dosing means known to the skilled person. A sublingual tablet may be placed under tongue, and the active ingredients absorbed through the surrounding mucous membranes.

In this respect, the compositions of the invention may be incorporated into various kinds of pharmaceutical preparations intended for transmucosal (e.g. sublingual) administration using standard techniques (see, for example, Lachman et al, “The Theory and Practice of Industrial Pharmacy”, Lea & Febiger, 3^{rd} edition (1986) and “Remington: The Science and Practice of Pharmacy”, Gennaro (ed.), Philadelphia College of Pharmacy & Sciences, 19^{th} edition (1995)).

Pharmaceutical preparations for sublingual administration may be obtained by combining compositions of the invention with conventional pharmaceutical additives and/or excipients used in the art for such preparations, and thereafter preferably directly compressed/compacted into unit dosage forms (e.g. tablets). (See, for example, Pharmaceutical Dosage Forms: Tablets. Volume 1, 2^{nd} Edition, Lieberman et al (eds.), Marcel Dekker, New York and Basel (1989) p. 354-356 and the documents cited therein.) Suitable compacting equipment includes standard tabletting machines, such as the Kilian SP300 or the Korsch EKO.

Suitable final sublingual tablet weights are in the range 30 to 400 mg, such as 50 to 200 mg, for example 60 to 180 mg, more preferably between about 70 and about 160 mg. Suitable final tablet diameters are in the range 4 to 10 mm, for example 5 to 9 mm, and more preferably about 6 to about 8 mm.

Irrespective of the foregoing, compositions of the invention should be essentially free (e.g. less than about 20% by weight based on the total weight of the formulation) of water. It will be evident to the skilled person that “premature” hydration will dramatically decrease the mucoadhesion promoting properties of a tablet formulation and may result in premature dissolution of the active ingredients.

Wherever the word “about” is employed herein in the context of dimensions (e.g. tablet sizes and weights, particle sizes etc.), surface coverage (e.g. of carrier particles by active ingredients), amounts (e.g. relative amounts of individual constituents in a composition or a component of a composition and absolute doses of active ingredients), it will be appreciated that such variables are approximate and as such may vary by ±10%, for example ±5% and preferably ±2% (e.g. ±1%) from the numbers specified herein.

Compositions of the invention may be administered by way of appropriate dosing means known to the skilled person. For example, a sublingual tablet may be placed under the tongue, and the active ingredients absorbed through the surrounding mucous membrane.

The compositions of the invention are useful in the treatment of pain for example the symptomatic treatment of pain, particularly severe, acute and/or breakthrough pain. According to a further aspect of the invention there is provided a method of treatment of pain which method comprises administration of a composition of the invention to a person suffering from, or susceptible to, such a condition.

For the avoidance of doubt, by “treatment” we include the therapeutic treatment, as well as the symptomatic treatment, the prophylaxis, or the diagnosis, of the condition.

The compositions of the invention enable the production of unit dosage forms that are easy and inexpensive to manufacture, and which enable the rapid release and/or a rapid uptake of the active ingredients employed through the mucosa, such as the oral mucosa, thus enabling rapid relief of pain symptoms, such as those described hereinbefore.

The compositions of the invention may also have the advantage that they substantially reduce the degree of absorption of active ingredients via swallowed saliva, as well as enabling the administration of “reduced” amounts of the active ingredients that are employed, so substantially reducing the risk of side effects, as well as intra- and interpatient variability of therapeutic response.

Compositions of the invention may also have the advantage that they may be prepared using established pharmaceutical processing methods and employ materials that are approved for use in foods or pharmaceuticals or of like regulatory status.

Compositions of the invention may also have the advantage that they may be more efficacious than, be less toxic than, be longer acting than, be more potent than, produce fewer side effects than, be more easily absorbed than, and/or have a better pharmacokinetic profile than, and/or have other useful pharmacological, physical, or chemical properties over, pharmaceutical compositions known in the prior art, whether for use in the treatment of pain or otherwise.

The invention is illustrated by way of the following examples.

### EXAMPLE 1

Fentanyl and ondansetron are firstly micronised and then accurately weighed out, along with the other excipients (see below), in appropriate proportions that enable the production of tablets with the absolute amounts of various ingredients mentioned below.

Pre-weighed quantities of the active ingredients and mannitol (Parteck M200; Merck, Germany) are then mixed in a Turbula mixer for 96 hours. Then, pre-weighed quantities of silicified microcrystalline cellulose (ProSolv; JRS Pharma, Germany) and sodium carboxymethylcellulose (Croscarmellose Sodium NF; Ac-Di-Sol®; FMC Corp., USA) are added and mixing is continued for 30 minutes. Finally, a pre-weighed quantity of magnesium stearate (Peter Greven, Netherlands) is added and mixing continued for another 2 minutes.

The powder mixture is then compacted using a single punch press (Korsch EKO) with 6 mm flat bevel edged punches, to produce tablets of a total weight of approximately 85 mg.

The absolute amounts of individual ingredients are as presented in the table below.

In-process controls are employed (tablet weight, crushing strength, friability and disintegration time), with test samples being withdrawn throughout the tabletting process. Tablets are packaged and labelled.

| Ingredient | Amount (mg) |
|---|---|
| fentanyl | 0.005 |
| ondansetron | 5.000 |
| mannitol | 65.000 |
| silicified microcrystalline cellulose | 10.000 |
| sodium carboxymethylcellulose | 4.000 |
| magnesium stearate | 1.000 |
| Total tablet weight | 85.005 |

### EXAMPLE 2

A butorphanol tablet composition is prepared in accordance with the procedure described in Example 1 above. The absolute amounts of individual ingredients are presented in the table below.

| Ingredient | Amount (mg) |
|---|---|
| butorphanol | 1.00 |
| ondansetron | 5.00 |
| mannitol | 20.00 |
| silicified microcrystalline cellulose | 18.00 |
| sodium carboxymethylcellulose | 35.00 |
| magnesium stearate | 1.00 |
| Total tablet weight | 80.00 |

### EXAMPLE 3

A nalbuphine tablet composition is prepared in accordance with the procedure described in Example 1 above. The absolute amounts of individual ingredients are presented in the table below.

| Ingredient | Amount (mg) |
|---|---|
| nalbuphine | 10.00 |
| ondansetron | 5.00 |
| mannitol | 35.00 |
| silicified microcrystalline cellulose | 9.00 |
| sodium carboxymethylcellulose | 40.00 |
| magnesium stearate | 1.00 |
| Total tablet weight | 100.00 |

1. A pharmaceutical composition for the treatment of pain comprising:
(a) a pharmacologically-effective amount of an opioid analgesic, or a pharmaceutically-acceptable salt thereof;
(b) a pharmacologically-effective amount of an antiemetic compound, or a pharmaceutically-acceptable salt thereof;
(c) a bioadhesion and/or a mucoadhesion promoting agent; and
(d) carrier particles,
wherein
(1) active ingredients (a) and (b) are presented in particulate form upon the surfaces of the carrier particles, which carrier particles are larger in size than the particles of the active ingredients; and
(2) the bioadhesion and/or mucoadhesion promoting agent is, at least in part, presented on the surfaces of the carrier particles.

2. A composition as claimed in claim 1, wherein the opioid analgesic is a naturally-occurring opium-derived compound, a semisynthetic derivative of an opium compound, or a synthetic compound with opioid or morphine-like properties.

3. A composition as claimed in claim 2, wherein the synthetic compound is a morphinan derivative, a benzomorphan derivative, a phenylpiperidine, a phenylheptamine, an open chain compound, a diphenylpropylamine derivative, a mixed agonist/antagonist or another synthetic opioid.

4. A composition as claimed in claim 2 or claim 3, wherein the opioid analgesic is selected from morphine, codeine, thebaine or a Diels-Alder adduct thereof, diamorphine, hydromorphone, oxymorphone, hydrocodone, oxycodone, etorphine, nicomorphine, hydrocodeine, dihydrocodeine, metopon, normorphine, N-(2-phenylethyl)normorphine, racemorphan, levorphanol, dextromethorphan, levallorphan, cyclorphan, butorphanol, nalbufine, cyclazocine, pentazocine, phenazocine, pethidine (meperidine), fentanyl, alfentanil, sufentanil, remifentanil, ketobemidone, carfentanyl, anileridine, piminodine, ethoheptazine, alphaprodine, betaprodine, 1-methyl-4-phenyl-1,2,3,6-tetrahydropyridine, diphenoxylate, loperamide, methadone, isomethadone, propoxyphene, levomethadyl acetate hydrochloride, dextromoramide, piritramide, bezitramide, dextropropoxyphene, buprenorphine, nalorphine, oxilorphan, tilidine, tramadol and dezocine.

5. A composition as claimed in claim 4, wherein the opioid analgesic is fentanyl.

6. A composition as claimed in claim 4, wherein the opioid analgesic is buprenorphine.

7. A composition as claimed in claim 1, wherein the antiemetic compound is selected from a phenothiazine, a 5-HT₃ antagonist, a dopamine receptor antagonist, an antihistamine, a piperazine derivative, butyrophenone, a cannabinoid, an antichlolinergic drug, cerium oxalate and ginger.

8. A composition as claimed in claim 7, wherein the antiemetic compound is a phenothiazine, an antihistamine or a 5-HT₃ receptor antagonist.

9. A composition as claimed in claim 7 or claim 8, wherein the antiemetic compound is ondansetron or granisetron.

10. A composition as claimed in claim 9, wherein the antiemetic compound is ondansetron.

11. A composition as claimed in claim 1, wherein the active ingredients (a) and (b) are in the form of microparticles.

12. A composition as claimed in claim 11, wherein the microparticles have a weight based mean diameter of less than about 15 μm.

13. A composition as claimed in claim 1, wherein the total amount of active ingredients (a) and (b) present is in the range about 0.1 to about 20% by weight based upon the total weight of the composition.

14. A composition as claimed in any one of the preceding claims, wherein the bioadhesion and/or mucoadhesion promoting agent is a polymeric substance with a weight average molecular weight above 5,000.

15. A composition as claimed in claim 14, wherein the bioadhesion and/or mucoadhesion promoting agent is selected from a cellulose derivative, a starch derivative, an acrylic polymer, polyvinylpyrrolidone, polyethylene oxide, chitosan, a natural polymer, scleroglucan, xanthan gum, guar gum, poly co-(methylvinyl ether/maleic anhydride) and crosscarmellose, or a mixture thereof.

16. A composition as claimed in claim 15, wherein the bioadhesion and/or mucoadhesion promoting agent is selected from hydroxypropylmethyl cellulose, hydroxyethyl cellulose, hydroxypropyl cellulose, methyl cellulose, ethyl hydroxyethyl cellulose, carboxymethyl cellulose, modified cellulose gum, sodium carboxymethyl cellulose, moderately cross-linked starch, modified starch, sodium starch glycolate, carbomer or a derivative thereof, crosslinked polyvinylpyrrolidone, polyethylene oxide, chitosan, gelatin, sodium alginate, pectin, scleroglucan, xanthan gum, guar gum, poly co-(methylvinyl ether/maleic anhydride) and crosscarmellose sodium, or a mixture thereof.

17. A composition as claimed in claim 16, wherein the bioadhesion and/or mucoadhesion promoting agent is crosscarmellose sodium or crosslinked polyvinylpyrrolidone.

18. A composition as claimed in claim 1 wherein the amount of bioadhesion and/or mucoadhesion promoting agent present is in the range of about 0.1 to about 25% by weight based upon the total weight of the composition.

19. A composition as claimed in claim 18, wherein the range is about 1 to about 15% by weight.

20. A composition as claimed in claim 1, wherein the carrier particle size is between about 50 and about 750 Tm.

21. A composition as claimed in claim 20, wherein the particle size is between about 100 and about 600 Tm.

22. A composition as claimed in claim 1, wherein the carrier particles comprise a carbohydrate, a pharmaceutically-acceptable inorganic salt or a polymer.

23. A composition as claimed in claim 22, wherein the particles comprise sugar, mannitol, lactose, sodium chloride, calcium phosphate, dicalcium phosphate hydrate, dicalcium phosphate dehydrate, tricalcium phosphate, calcium carbonate, barium sulfate, microcrystalline cellulose, cellulose, crosslinked polyvinylpyrrolidone or a mixture thereof.

24. A composition as claimed in claim 23, wherein the particles comprise mannitol and/or lactose.

25. A composition as claimed in claim 1 wherein the bioadhesion and/or mucoadhesion promoting agent has a particle size in the range of about 1 to about 100 Tm.

26. A composition as claimed in claim 1, wherein the relative sizes and amounts of the particles of active ingredients and the carrier particles that are employed are sufficient to ensure that the carrier particles may be at least about 90% covered by the active ingredients.

27. A composition as claimed in claim 1 which is in the form of a tablet suitable for sublingual administration.

28. A composition as claimed in claim 27, wherein the composition further comprises a disintegrating agent.

29. A composition as claimed in claim 28, wherein the disintegrating agent is selected from crosslinked polyvinylpyrrolidone, carboxymethyl starch, natural starch and mixtures thereof.

30. A composition as claimed in claim 28 or claim 29, wherein the amount of disintegrating agent is between about 2 and about 7% by weight based upon the total weight of the composition.

31. A process for the preparation of a composition as defined in claim 1, which comprises:
(i) dry mixing carrier particles with the active ingredients (a) and (b); and
(ii) admixing bioadhesion and/or mucoadhesion promoting agent with carrier particles.

32. A process for the preparation of a sublingual tablet as defined in claim 27, which comprises directly compressing or compacting a composition as defined in any one of claims 1 to 26.

33. (canceled)

34. A method of treatment of pain which method comprises administration of a composition as defined in claim 1 to a patient suffering from, or susceptible to, such a condition.

35. A method as claimed in claim 34, wherein the pain is severe, acute and/or breakthrough pain.
